(19) United States
(12) Patent Application Publication
(10) Pub. No.: US 2018/0116700 A1
(43) Pub. Date: May 3, 2018

(54) Craniomaxillofacial Bone Plate Sizers and Templates

(71) Applicant: KLS-Martin, L.P., Jacksonville, FL (US)

(72) Inventors: Thomas S. Johnston, JR., Jacksonville, FL (US); Jennifer Pinto, Jacksonville, FL (US); Klaus Kohler, Muhlheim (DE)

(73) Assignee: KLS-Martin, L.P., Jacksonville, FL (US)

(21) Appl. No.: 15/800,459

(22) Filed: Nov. 1, 2017

Related U.S. Application Data
(60) Provisional application No. 62/415,940, filed on Nov. 1, 2016.

(51) Int. Cl. A61B 17/80

(52) U.S. Cl. CPC A61B 17/8061; A61B 17/688; A61B 17/8085

(57) ABSTRACT

One or more arrays of flexible, disposable, bone plate sizers with corresponding arrays of permanent bone plates, systems using these bone plate sizers and/or templates, and methodologies using these bone plate sizers and/or templates are embodied. Each array of disposable bone plate sizers has a plurality of sizers having differing configurations and sizes, each of the disposable sizers in a given array corresponding to a particular bone screw size. Each of the disposable bone plate sizers in a given array are mounted upon and easily detachable from a frame member. The disposable bone plate sizers may be malleable such that may be manipulated by the surgeon into the desired three-dimensional shape to match the contour of the bone segments to be joined, thereby providing a template to shape the corresponding permanent bone plate.

### BACKGROUND OF THE INVENTION

This invention relates generally to the field of bone plates used in osteosynthesis, the plates being of the type affixed to and between bone segments to secure and fix the bone segments against relative moment such that bone growth results in a joining of the bone segments. More particularly, the invention relates to craniomaxillofacial bone plates, systems and methodologies.

It is often necessary or desirable to join separated bone segments, the bone segments being separated because of malformation, accident or intentional surgical osteotomies. Bone plates made of a biocompatible metal possessing sufficient structural properties to fix the bone segments, such as for example titanium, stainless steel or aluminum, are well known in the art. The bone plates typically comprise elongated, thin members having apertures designed to receive biocompatible bone screws therethrough such that the plates may be mechanically secured to multiple bone segments across a separation line or gap.

The human craniomaxillofacial structure is relatively uniform, such that craniomaxillofacial bone plates are often produced in sets of individual members, each set being sized for use with a particular size of bone screw. The bone plates may be pre-contoured into three-dimensional configurations or may be presented in planar configurations that allow the surgeon to bend and shape a given plate to the desired three-dimensional configuration needed to match the contour and shape of the bone segments. A set of planar bone plates will include multiple configurations, such as linear, curved-in-plane, T-shaped, etc., and similar configuration plates will vary in their lengths, as well as the number and spacing of screw-receiving apertures. Sets are typically provided in protective, sterilizable cases.

Because the surgeons need to physically match the individual bone plates to the bone segments prior to affixation, and typically will need to try multiple plates before determining the best fit, problems occur with maintaining the sterility of all the plates in an array. Furthermore, the need to bend or shape the plates to provide for a precise fit may result in crimped, wrinkled or weakened plates due to repeated attempts to achieve the proper fit, which will be detrimental to the long-term efficacy of the bone plates.

It is an object of this invention to address the problems set forth above by providing sterilized arrays of disposable, flexible, bone plate sizers and/or templates in combination with corresponding arrays of matching permanent bone plates, wherein the sizers of a particular array are removably attached to a frame member and the corresponding bone plates may be individual members presented in a case or removably attached to a second frame member. The surgeon is then able to choose the proper configuration and size of the bone plate by first handling, positioning and possible shaping disposable sizers without concern for loss of sterility from coming into contact with the patient. Upon determination of the properly sized disposable sizer, the surgeon knows which of the permanent bone plates to utilize. In certain embodiments, the disposable sizers may be malleable to allow the surgeon to achieve three-dimensional conformation to the patient's bone structure, thereby providing a template for shaping the corresponding permanent bone plate prior to affixation to the patient.

### SUMMARY OF THE INVENTION

The invention is in general embodied as a kit or combination of at least one array of disposable, flexible, bone plate sizers and/or templates and a corresponding array or arrays of permanent bone plates, systems using these sizers and bone plates, and methodologies using these sizers and bone plates, the bone plates matching. Each array of disposable bone plate sizers comprises a plurality of individual sizers having differing configurations and sizes, but related such as by all receiving a particular size of bone screw, each of the disposable sizers removably mounted to a frame. The corresponding array of bone plates has individual bone plates matching the sizers, such that one sizer is paired with one bone plate having matching size and configuration. The disposable bone plate sizers are preferably made of plastic, but may be made of metal. Each of the disposable bone plate sizers in a given array are easily detachable from the frame member by bending, cutting or similar methods. The disposable bone plate sizers may be malleable such that may be manipulated by the surgeon into the desired three-dimensional shape to match the contour of the bone segments to be joined, thereby providing a template to shape the corresponding permanent bone plate prior to affixation. The sizers may be provided with marking apertures and/or identifying indicia.

Tin alternative language, the invention may be summarized as the combination comprising an array of individually sized or configured bone plates, each said bone plate in said bone plate array having a different size or configuration, and an array of individually sized or configured bone plate sizers removably mounted to a sizer frame member, each said bone plate sizer in said sizer array having a different size or configuration, wherein for each said bone plate there is a paired said bone plate sizer of matching size and configuration. And furthermore, wherein said bone plates are removable mounted to a bone plate frame member; wherein each of said bone plates of said bone plate array are configured to receive one size bone screw; wherein each of said bone plate sizers comprises mounting segments connected by bridging members, and wherein each of said bone plates comprises mounting segments connected by bridging members and screw-receiving apertures positioned in said mounting segments; wherein said bone plate sizers are malleable; and/or wherein said sizers further comprise marking apertures disposed in said mounting segments.

Alternatively, a kit comprising at least one array of individually sized or configured bone plates, each said bone plate in said bone plate array having a different size or configuration, and an array of individually sized or configured bone plate sizers removably mounted to a sizer frame member, each said bone plate sizer in said sizer array having a different size or configuration, wherein for each said bone plate there is a paired said bone plate sizer of matching size and configuration; and/or further wherein said bone plates are removable mounted to a bone plate frame member; wherein each of said bone plates for a single said at least one bone plate array are configured to receive one size bone screw; wherein each of said bone plate sizers comprises mounting segments connected by bridging members, and wherein each of said bone plates comprises mounting segments connected by bridging members and screw-receiving apertures positioned in said mounting segments; wherein said bone plate sizers are malleable; and/or wherein said sizers further comprise marking apertures disposed in said mounting segments.

Alternatively, a kit comprising an array of individually sized or configured bone plate sizers removably mounted to a first frame member, each said bone plate sizer having a different size or configuration, and an array of individually sized or configured bone plates, each said bone plate sizer having a different size or configuration, wherein said bone plate sizers match the size and configurations of said bone plates; wherein each of said bone plates of said bone plate array are configured to receive one size bone screw; wherein each of said bone plate sizers comprises mounting segments connected by bridging members, and wherein each of said bone plates comprises mounting segments connected by bridging members and screw-receiving apertures positioned in said mounting segments; and/or wherein said bone plate sizers are malleable.

### BRIEF DESCRIPTION OF THE DRAWINGS

FIG. 1 shows one embodiment of an array of disposable bone plate sizers removably mounted on a frame.

FIG. 2 shows a corresponding array of bone plates removably mounted on a frame, the bone plates corresponding in size and configuration to the sizers of FIG. 1.

FIG. 3 shows an alternative embodiment of an array of disposable bone plate sizers removably mounted on a frame, the sizers having marking apertures.

FIG. 4 shows a corresponding array of bone plates removably mounted on a frame, the bone plates corresponding in size and configuration to the sizers of FIG. 3.

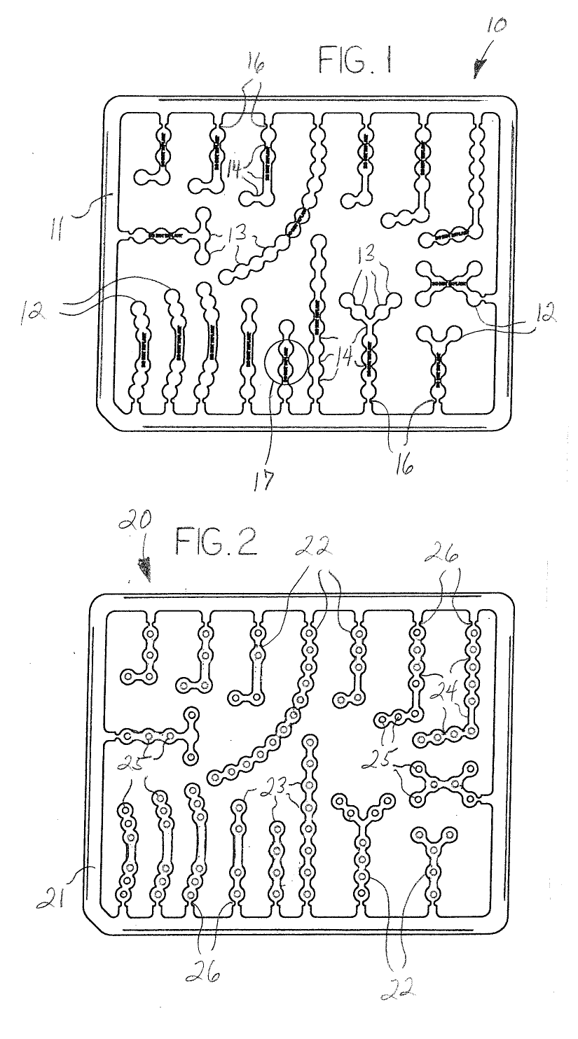

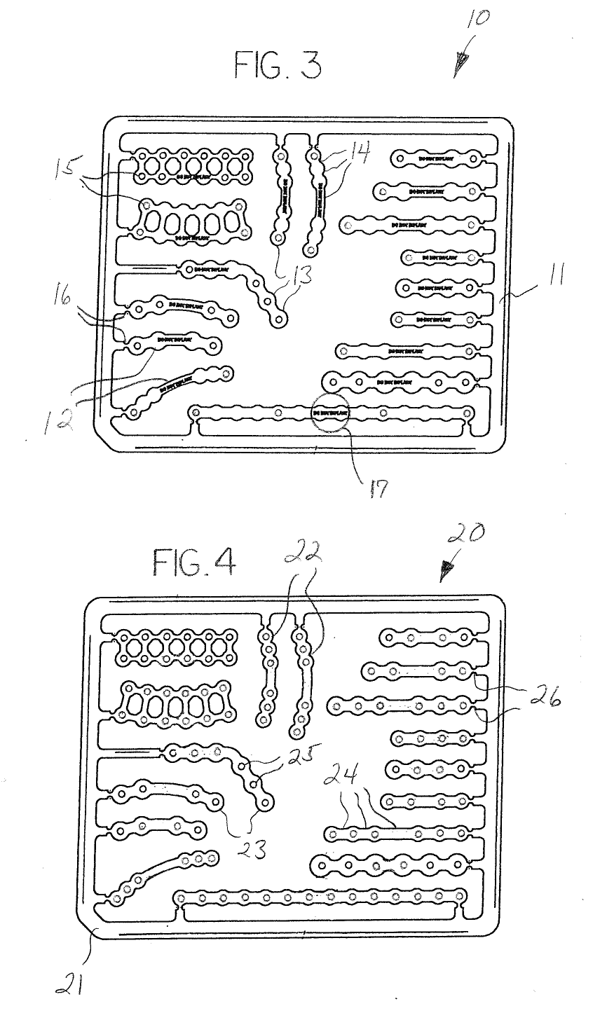

### DETAILED DESCRIPTION OF THE INVENTION

The invention in its various embodiments will now be described in detail with reference to the drawings, the drawings being provided for descriptive and illustrative purposes, such that they are not meant to be limiting as to the scope of the claims.

The invention is in various embodiments a kit or combination of an array or multiple arrays 10 of sterile or sterilizable, disposable, bone plate sizers 12, preferably flexible, removably mounted on a sizer frame 11, each of the bone plate sizers 12 in a given array 10 varying in configuration and size, each sizer array 10 being in combination with a corresponding and matching array 20 of permanent bone plates 22 adapted for affixation to separated bone segments in order to preclude relative movement of the segments to enable osteosynthesis to occur to join the segments. In alternative embodiments, the bone plate sizers 12 may be malleable such that they may be manipulated into three-dimensional shapes required to conform to the contours of the bone segments, the sizers 12 thus defining templates for the surgeon to use in shaping the permanent bone plates 22. As used herein, the term “malleable” shall be taken to define the characteristic or property wherein the bone plate sizers 12 retain a post-manipulation shape, while the term “flexible” shall be taken to define the characteristic or property wherein the bone plate sizers 12 are substantially or completely resilient, such that post-manipulation the sizers 12 will return near or completely to their original configuration or shape.

The bone plate sizers 12 are preferably composed of a synthetic polymer material, such as for example PETP, POM, PPSU, PEEK, PE, PET, PA, PPS, PTFE or PVDF, but may be composed of a metal, such as for example aluminum, titanium or stainless steel. The sizers 12 should be formed of a biocompatible material that may be sterilized. Each of the sizers are 12 removably mounted to a sizer frame 11, preferably at one or more attachment points, i.e., areas of diminished dimension such that an individual sizer 12 may be easily removed by breaking, bending, twisting, cutting, etc. This enables individual sizers 12 to be removed from the sizer frame 11 as needed. For example, the sizers 12 may be connected to the frame 11 by relatively narrow or thin neck members 16, such that a sizer 12 may be removed from the frame 11 by twisting, breaking or cutting the neck member or members 16. An array of sizers 12 may be produced with various manufacturing techniques, such as for example milling, injection molding, laser cutting, water jet cutting, erosion, etching, additive manufacturing, etc. The shape of the sizer frame 11 may vary, such as for example circular, rectangular, square, linear or curved, and the sizers 12 may be positioned in the interior of the frame 11, on the exterior of the sizer frame 11, or both.

Individual sizers 12 in an array will vary in configuration and size, such that they may be linear, curved-in-plane, double-rowed, T-shaped, cross-shaped or of any other configuration. The sizers 12 may be formed in three-dimensional, i.e., non-planar, configurations as well. A sizer 12 preferably comprises an elongated or branched main body having wider segments referred to herein as mounting segments 13 connected together by narrower bridging members 14, relatively speaking. Marking apertures 16 may or may not be provided in the mounting segments 13 of the sizers 12, such that the marking apertures 16 may be used to mark the proper location to receive bone screws to secure the bone plates 22 to the bone. Alternatively, the marking apertures 16 may be utilized to temporarily affix the sizer 12 to a bone using a mechanical fastener is such is useful in locating or manipulating the sizer 12. Visible identifying indicia 17 may be provided on the sizers 12, the indicia 17 indicating for example the bone screw size for that particular sizer or a label such as “DO NOT IMPLANT” to ensure that the surgeon does not affix a sizer 12 to the patient instead of a bone plate 22.

A sterile or sterilizable permanent bone plate 22 typically comprises an elongated or branched main body having broad or widened mounting segments 23 connected by narrower bridging members 24, relatively speaking, with screw-receiving apertures 25 disposed in the mounting segments 23. The array of bone plates 22 may be provided as individual discrete members of varying configurations and sizes retained within a housing or case. Alternatively, as with the sizers 12, the array of bone plates 22 may be connected to a bone plate frame member 21 at one or more attachment points, such as neck members 26, for easy removal from the frame member 21 by breaking, bending, twisting, cutting, etc.

A sizer/bone plate kit comprises one or more arrays of bone plates 20 and sizers 10, wherein each bone plate 22 of array 20 has a matching and corresponding sizer 12 of array 10, the sizers 12 and the bone plates 22 in the corresponding pairing of arrays 10/20 matching in configuration and size. Thus, for example, a bone plate array 20 having a three T-shaped, three L-shaped and three Y-shaped bone plates 22 will have a corresponding sizer array 10 having three T-shaped, three L-shaped and three Y-shaped sizers 12 of equal size and dimension to the bone plates 22 of bone plate array 20, thereby defining a total of nine pairs of matching sizers 12 and bone plates 22.

Craniomaxillofacial bone screws come in various sizes, ranging for example from 1.0 mm screws used in neuro and midface applications, 1.5 mm screws used in non-load bearing cranial and midface applications, 2.0 mm screws used in orthognathic midface and mandible trauma applications, to 2.3 and 2.7 mm locking screws used in mandible applications. Thus, a surgical kit may comprise a first combination of sizer/bone plate arrays 10a/20a with sizer/bone plate pairs adapted for 1.0 mm screws, a second combination of sizer/bone plate arrays 10b/20b with sizer/bone plate pairs adapted for 1.5 mm screws, a third combination of sizer/bone plate arrays 10c/20c with sizer/bone plate pairs adapted for use with 2.0 mm screws, etc.

The sizers 12 and their corresponding permanent bone plates 22 may define mandibular angle plates, zygomatic arch plates, glabellar plates, medial canthal plates, nasofrontal plates, lateral buttress plates, forehead plates, panskull plates, frontozygomatic suture plates, inferior orbital rim plates, medial buttress J plates, medial buttress T plates, L plates, symphysis plates, interior body plates, angle plates, ridge plates, posterior body plates, hemimandible plates, etc. Thus, the sizers 12 and corresponding permanent bone plates 22 may be presented on paired, corresponding frames 11/21 as a kit of frontal group sizers/plates 10d/20d, a kit of zygomatic group sizers/plates 10e/20e, a kit of maxillary group sizers/plates 10e/20e, or a kit of mandibular group sizers/plates 10f/20f, for example.

The sizers 12 may be resiliently flexible, or more preferably, may be malleable at ambient temperature such that the surgeon may bend the sizer 12 into a three-dimensional configuration to match the contours of the bone segments to be joined. The sizers 12 may alternatively be formed of a rigid material that becomes malleable upon heating or chemical application, whereby after being properly shaped the sizer 12 hardens back into a rigid status. After manipulation, malleable sizers 12 will then define templates which provide the surgeon with a visual indication of the proper three-dimensional configuration for the bending of the permanent bone plate 22 to be affixed to the bone segments.

The surgeon, upon determining the optimum screw size for the bone segments to be stabilized, identifies the proper array 20 of bone plates 22 and the corresponding array 10 of sizers 12 to be utilized. Individual sizers 12 are then removed from the frame 11 and manually positioned or applied to the bone segments to deteimine the optimum configuration and size of bone plate 22. If the sizer 12 is malleable, the surgeon may bend the sizer 12 as needed into the proper three-dimensional configuration to use as a template. With knowledge of the proper sizer 12, the corresponding permanent bone plate 22 is chosen from the corresponding array 20 of permanent bone plates 22, shaped if necessary to match the three-dimensional configuration of the sizer 12, and then permanently affixed to the patient using bone screws. The disposable sizers 12 are then discarded.

It is understood that equivalents and substitutions for certain elements described above may be obvious to those of skill in the art, and therefore the true scope and definition of the invention is to be as set forth in the following claims.

We claim:

1. The combination comprising an array of individually sized or configured bone plates, each said bone plate in said bone plate array having a different size or configuration, and an array of individually sized or configured bone plate sizers removably mounted to a sizer frame member, each said bone plate sizer in said sizer array having a different size or configuration, wherein for each said bone plate there is a paired said bone plate sizer of matching size and configuration.

2. The combination of claim 1, wherein said bone plates are removable mounted to a bone plate frame member.

3. The combination of claim 1, wherein each of said bone plates of said bone plate array are configured to receive one size bone screw.

4. The combination of claim 2, wherein each of said bone plates of said bone plate array are configured to receive one size bone screw.

5. The combination of claim 1, wherein each of said bone plate sizers comprises mounting segments connected by bridging members, and wherein each of said bone plates comprises mounting segments connected by bridging members and screw-receiving apertures positioned in said mounting segments.

6. The combination of claim 2, wherein each of said bone plate sizers comprises mounting segments connected by bridging members, and wherein each of said bone plates comprises mounting segments connected by bridging members and screw-receiving apertures positioned in said mounting segments.

7. The combination of claim 3, wherein each of said bone plate sizers comprises mounting segments connected by bridging members, and wherein each of said bone plates comprises mounting segments connected by bridging members and screw-receiving apertures positioned in said mounting segments.

8. The combination of claim 4, wherein each of said bone plate sizers comprises mounting segments connected by bridging members, and wherein each of said bone plates comprises mounting segments connected by bridging members and screw-receiving apertures positioned in said mounting segments.

9. The combination of claim 1, wherein said bone plate sizers are malleable.

10. The combination of claim 2, wherein said bone plate sizers are malleable.

11. The combination of claim 5, wherein said sizers further comprise marking apertures disposed in said mounting segments.

12. The combination of claim 6, wherein said sizers further comprise marking apertures disposed in said mounting segments.

13. A kit comprising at least one array of individually sized or configured bone plates, each said bone plate in said bone plate array having a different size or configuration, and an array of individually sized or configured bone plate sizers removably mounted to a sizer frame member, each said bone plate sizer in said sizer array having a different size or configuration, wherein for each said bone plate there is a paired said bone plate sizer of matching size and configuration.

14. The kit of claim 13, wherein said bone plates are removable mounted to a bone plate frame member.

15. The kit of claim 13, wherein each of said bone plates for a single said at least one bone plate array are configured to receive one size bone screw.

16. The kit of claim 13, wherein each of said bone plate sizers comprises mounting segments connected by bridging members, and wherein each of said bone plates comprises mounting segments connected by bridging members and screw-receiving apertures positioned in said mounting segments.

17. The kit of claim 13, wherein said bone plate sizers are malleable.

18. The kit of claim 16, wherein said sizers further comprise marking apertures disposed in said mounting segments.

19. A kit comprising an array of individually sized or configured bone plate sizers removably mounted to a first frame member, each said bone plate sizer having a different size or configuration, and an array of individually sized or configured bone plates, each said bone plate sizer having a different size or configuration, wherein said bone plate sizers match the size and configurations of said bone plates; wherein each of said bone plates of said bone plate array are configured to receive one size bone screw; wherein each of said bone plate sizers comprises mounting segments connected by bridging members, and wherein each of said bone plates comprises mounting segments connected by bridging members and screw-receiving apertures positioned in said mounting segments.

20. The kit of claim 19, wherein said bone plate sizers are malleable.
